(19) United States
(12) Patent Application Publication
(10) Pub. No.: US 2022/0175750 A1
(43) Pub. Date: Jun. 9, 2022

(54) SUSTAINED RELEASE FORMULATION FOR LOCAL DELIVERY OF CDK9 INHIBITORS

(71) Applicants: The Regents of the University of California, Oakland, CA (US); Tesio Pharmaceuticals, Inc., Davis, CA (US)

(72) Inventors: Dominik R. HAUDENSCHILD, Oakland, CA (US); Jasper H. N. YIK, Oakland, CA (US); Jamal S. LEWIS, Oakland, CA (US); Tom YARBROUGH, Davis, CA (US)

(21) Appl. No.: 17/049,798

(22) PCT Filed: Apr. 23, 2019
(86) PCT No.: PCT/US2019/028721

§ 371 Date: Oct. 22, 2020

(51) Int. Cl. A61K 31/453; A61K 9/50; A61K 31/4025; A61K 31/519; A61P 19/02

(52) U.S. Cl. CPC A61K 31/453; A61K 9/5031; A61P 19/02; A61K 31/519; A61K 31/4025

(57) ABSTRACT

The present disclosure describes a novel sustained-release formulation for the local delivery of CDK9 inhibitors.

### CROSS-REFERENCES TO RELATED APPLICATIONS

This application is a U.S. National Phase Application of PCT International Application No. PCT/US2019/028721, filed Apr. 23, 2019, which is an International Application of and claims the benefit of priority to U.S. Patent Application No. 62/661,599, filed on Apr. 23, 2018, each of which is incorporated by reference herein in its entirety.

### STATEMENT AS TO RIGHTS TO INVENTIONS MADE UNDER FEDERALLY SPONSORED RESEARCH OR DEVELOPMENT

This invention was made with Government support under National Institutes of Health (NIH) Grant No. R21AR063348 and ARMY/MRMC Grant No. W81XWH-12-1-0311. The Government has certain rights in the invention.

### BACKGROUND OF THE INVENTION

Recent advances conclusively show that the general transcription factor P-TEFb, comprising cyclin-dependent kinase 9 (CDK9) and cyclin T, controls the rate-limiting step for activation of all primary response genes. The primary response genes (PRGs) are genes that need to be immediately activated at the transcriptional level in response to acute changes in the environment. PRGs include the typical inflammatory genes (IL-1, TNF, IL-6, iNOS, etc), as well as other cell-type specific genes. In the event of an acute change in the cellular environment (for example an injury to a joint), the transcription of PRGs requires the activity of CDK9. Thus CDK9 is a novel target for therapies designed to limit cellular responses to an acute event such as an injury. Small-molecule CDK9 inhibitors exist, however they diffuse rapidly, generally have short in-vivo half-lives, and systemic administration can cause undesired off-target effects.

### BRIEF SUMMARY OF THE INVENTION

Provided herein are microparticle formulations of CDK9 inhibitors that provide sustained release of the inhibitors locally to affected tissues while avoiding unwanted systemic effects. The formulations of the invention comprise CDK9 inhibitors encapsulated in microparticles of poly(lactic-co-glycolic) acid (PLGA). Surprisingly, we found that many combinations of microparticle components failed to provide an adequate sustained release, resulted in microparticles of an unacceptably large average size, failed to release at least about 80% of the encapsulated drug, and/or encapsulated an inadequate amount of CDK9 inhibitor.

The microparticle formulations herein provide a sustained release of the CDK9 inhibitor over a period of about 4 to about 6 weeks, wherein a limited amount is released within the first 24 hours following administration.

The microparticles of the invention comprise a CDK9 inhibitor and a PLGA polymer, ranging in average size from about 2 microns to about 150 microns.

Further provided herein is a pharmaceutical composition comprising a plurality of microparticles of the disclosure, and a pharmaceutically acceptable carrier.

Also provided herein is a method of treating a subject, e.g., a human or veterinary subject, suffering from a disease or disorder in an articular joint, the method comprising injecting into the articular joint a therapeutically effective amount of the pharmaceutical composition.

One aspect of the invention is a microparticle comprising a cyclin-dependent kinase 9 (CDK9) inhibitor and poly(lactic-co-glycolic) acid (PLGA), wherein the CDK9 inhibitor is encapsulated by the PLGA, and wherein the microparticle provides a sustained release of the CDK9 inhibitor.

Another aspect of the invention is a pharmaceutical composition comprising a plurality of microparticles of the invention, and a pharmaceutically acceptable carrier.

Another aspect of the invention is a method of treating a subject in need thereof, comprising administering a therapeutically effective amount of a plurality of microparticles, the microparticles comprising a CDK9 inhibitor and a poly(lactic-co-glycolic) acid (PLGA), wherein the CDK9 inhibitor is encapsulated by the PLGA, and wherein the microparticles provide a sustained release of the CDK9 inhibitor.

Another aspect of the invention is a method of treating a subject in need thereof, comprising administering a pharmaceutical composition comprising a plurality of microparticles of the invention and a pharmaceutically acceptable carrier.

Another aspect of the invention is a method of treating a site of inflammation comprising, administering to the site a composition comprising a CDK9 inhibitor formulated into a plurality of microparticles, wherein the microparticles provide a sustained release of the CDK9 inhibitor at the site for at least 24 hours, and whereby inflammation at the site is thereby reduced or ameliorated.

### BRIEF DESCRIPTION OF THE DRAWINGS

FIG. 1 shows the effect on gene expression in primary human chondrocytes in monolayer culture, treated with 10 ng/mL IL-1β with or without 300 nM flavopiridol for 5 hours. CDK9 inhibition effectively suppresses the transcription of primary inflammatory response genes.

FIGS. 2A, 2B, 2C, and 2D show that CDK9 inhibition by systemic administration of flavopiridol effectively suppresses the transcription of primary response genes upon ACL-rupture in mice. FIG. 2A shows the increase in IL-1β mRNA expression with and without flavopiridol. FIG. 2B shows the increase in IL-6 mRNA expression with and without flavopiridol. FIG. 2C shows the increase in MMP-13 mRNA expression with and without flavopiridol. FIG. 2D shows the increase in ADAMTS4 mRNA expression with and without flavopiridol.

FIGS. 3A, 3B, 3C, and 3D CDK9 inhibition with repeated systemic administration of flavopiridol effectively suppresses the transcription of primary response genes upon ACL-rupture in mice. A window of at least 3 hours exists after injury during which CDK9 inhibition with flavopiridol is effective at preventing transcription of primary response genes. FIG. 3A shows the increase in IL-1β gene expression after 0, 1, or 2 administrations of flavopiridol. FIG. 3B shows the increase in IL-6 gene expression after 0, 1, or 2 administrations of flavopiridol. FIG. 3C shows the increase in IL-1β gene expression after a delay of 0, 1, 2, or 3 hours before flavopiridol administration (the left-most bar shows expression without flavopiridol treatment). FIG. 3D shows the increase in IL-6 gene expression after a delay of 0, 1, 2, or 3 hours before flavopiridol administration (the left-most bar shows expression without flavopiridol treatment).

FIG. 4 Typical size distribution of PLGA microparticles containing flavopiridol, prepared in Example 1. FIG. 4A graphically depicts the size distribution of PLGA microparticles of lot 53024, with a table of measurements. FIG. 4B is a light microscope image of microparticles of the invention. FIG. 4C is a light microscope image of microparticles of the invention in a different formulation. FIG. 4D is a light microscope image of microparticles of the invention in still another formulation.

FIG. 5: The in vitro release profile of flavopiridol from a batch of PLGA microparticles (Example 1) in 1% Tween® 20 in 1×PBS shows nearly linear release kinetics of the flavopiridol from the microparticles out to 30 days, with approximately 90% of the drug released.

FIG. 6: Flavopiridol was encapsulated at 1% w/v ratio in PLGA microparticles (MPs) synthesized using Purasorb® PDLG 5004A (Corbion) in methylene chloride at 20% w/v ratio and vortexed vigorously for 30 seconds after addition of polyvinyl alcohol (10%) at 2:1 ratio. The solution was then added dropwise to 1% PVA and mixed overnight, with 3× washes next day and lyophilized overnight. The average loading efficiency of 51.5% was determined by dissolving flavopiridol PLGA MPs in DMF, measuring flavopiridol concentration from a standard curve, and comparing to the starting amounts (n=3 for all data points). FIG. 6A: Using an AccuSizer Optical Particle Sizer Model 770, the size of the flavopiridol MPs were determined to have an average diameter of 6.87 μM (blank MPs had an average diameter of 10.77 μm). FIG. 6B: Linear standard curve for different concentrations of flavopiridol in solution of dissolved PLGA in DMSO acquired with Nanodrop 2000 Spectrophotometer at 274 nm. FIG. 6C: Release kinetics of flavopiridol loaded MPs over 42 days in 1% Tween® PBS solution demonstrating near linear release. FIG. 6D: SEM image of flavopiridol MPs using a Phillips XL30 Microscope.

FIG. 7 Intra-articular injection of sustained-release PLGA-Flavopiridol protected the knee joint from OA for at least 3 weeks in a ACL-rupture PTOA rat model. FIG. 7A shows the localized MMP expression in the untreated injured knee, as compared with the treated and control knees. FIG. 7B: Flavopiridol-PLGA microparticles were administered by IA injection in rats with ACL-rupture injury (triangles), and empty PLGA microparticles without drug were control (squares). Joint MMPSense activity was repeatedly measured using the MMPSense 750 reagent, and the activity in the injured leg normalized to that in the uninjured contralateral leg of the same animal. A ratio of 1.0 indicates no effect of the injury. These results demonstrate that the Flavopiridol-releasing microparticles protected against the activation of cartilage-degrading MMP enzymes for at least 3 weeks.

FIG. 8 shows that the flavopiridol released from PLGA microparticles retains its potency, using a cell-based assay. FIG. 8A: As shown in lanes 3-5 of the graph, flavopiridol prevented over 99% of the IL-1β response observed with IL-1 stimulation (lane 2) as measured by a luciferase reporter gene, and this was consistent between flavopiridol before (lane 5) and after PLGA encapsulation (lanes 3, 4). The stimulus is IL-1β treatment, which causes transcriptional activation of many Primary Response Genes. The readout of luciferase activity is driven by a NFκB-responsive promoter, which serves as a Primary Response Gene that can easily be quantified. Baseline values in the control are low, <5000. This IL-1β-induced increase is not observed when flavopiridol is present, and importantly, flavopiridol released from two different PLGA formulations (53010 and 53012) is as active as flavopiridol before PLGA encapsulation (Flavo). Note the Log scale on the Y axis of FIG. 8A. FIG. 8B: the lower graph is on a linear Y-axis, showing that flavopiridol prevented between 99.6% and 99.8% of IL-1β response, and PLGA-encapsulated flavopiridol retains its potency.

FIG. 9 shows a PLGA-Flavopiridol formulation that did not meet specification because the microparticles formed large aggregates. In this case the microparticles with PLGA-encapsulated flavopiridol formed aggregates of 100-200 microns, as shown by the size distribution graph. These particles exhibited clumping (see Example 6 below, Table 3 (formulations E and H).

FIG. 10 shows PLGA-flavopiridol formulations that did not meet specifications due to incomplete flavopiridol release. This graph shows two different formulations of PLGA-Flavopiridol that did not meet specifications because they show incomplete release of flavopiridol<80%. These formulations correspond to Formulations J and L (see Example 6 below, Table 3) after treatment with gamma irradiation, a treatment that can be used in some instances for sterilization, but here unfavorably effects the release characteristics of the flavopiridol.

FIG. 11 shows PLGA-flavopiridol formulations that did not meet specifications because of non-linear flavopiridol release (initial burst followed by almost no additional release). The graph shows two formulations of PLGA-flavopiridol that did not meet specifications because they have: (a) an initial burst of flavopiridol release; (b) very slow flavopiridol release after initial burst. (see Example 6 below, Table 3, formulations M and N).

### DETAILED DESCRIPTION OF THE INVENTION

### I. General

In an embodiment, this invention describes a novel sustained-release formulation for the local delivery of CDK9 inhibitors, in which the inhibitor is encapsulated in bioresorbable polymers, and is released over time as the polymer degrades. The inhibitor is locally available at therapeutic levels over a prolonged period of time, while minimizing the overall systemic dose.

### II. Definitions

“PLGA” is poly(lactic-co-glycolic) acid.

“CDK” refers to cyclin-dependent kinase. CDK9 is cyclin-dependent kinase 9.

“IL” is interleukin.

“TNF” is tumor necrosis factor.

“MMP” is matrix metalloproteinase.

“ACL” is the anterior cruciate ligament.

“PTOA” is post-traumatic osteoarthritis.

A “derivative” of a CDK9 inhibitor is an ester, amide, or prodrug of the CDK9 inhibitor, where the ester, amide, or prodrug substituent is cleaved or hydrolyzed after administration to a subject.

The term “microparticle” refers to a PLGA particle have a diameter between about 0.5 μm and about 100 μm.

The term “sustained release” refers to the release of CDK9 inhibitor over an extended period of time after administration, generally between about 1 hour and about 30-60 days.

The term “inherent viscosity” (abbreviated herein as “IV”) refers to a property of the polymers used in the present invention. Inherent viscosity, ηᵢ, is calculated from the equation ηᵢ=(In ηᵣ)/c, where “c” is the concentration of the polymer in solution, and ηᵣ is the relative viscosity. The relative viscosity in turn is given by ηᵣ=η/η₀, where η is the measured viscosity, and η₀ is the viscosity of the solvent. The IV can be extrapolated to 0 concentration, the result of which is termed the “intrinsic viscosity” (“[η]”), which correlates with the molecular weight of the polymer. Thus, IV is an indication of the molecular weight of the polymer. IV is expressed in units of deciliter per gram, dL/g. Viscosity is commonly measured by means of a viscometer, for example a rotational viscometer, tuning fork vibration viscometer, glass capillary viscometer, falling ball viscometer, or the like. The IV for polymers used in the instant invention can be determined using a glass capillary viscometer, with the polymer dissolved in chloroform or hexafluoroisopropanol (HFIP).

The term “subject” as used herein refers to a mammal, which can be a human or a non-human mammal, for example a companion animal, such as a dog, cat, rat, or the like, or a farm animal, such as a horse, donkey, mule, goat, sheep, pig, or cow, and the like.

The term “therapeutically effective amount” refers to the amount of the microparticles of the invention sufficient to suppress undesirable inflammation and to eliminate or at least partially arrest symptoms and/or complications. Specifically, a therapeutically effective amount is the amount sufficient to suppress expression of primary response genes such as IL-1β and IL-6 to no more than 50%, 40%, 30%, 20%, 10%, 5%, or 1% of the otherwise expected gene activity. Amounts effective for this use will depend on, e.g., the inhibitor composition, the manner of administration, the stage and severity of the disease being treated, the weight and general state of health of the patient, and the judgment of the prescribing physician. In practice, the amount of CDK9 inhibitor required for a therapeutic effect in the method of the invention will be less than the amount required for systemic administration, due to the local nature of the microparticle drug release. The microparticles of the invention can be administered chronically or acutely to reduce, inhibit or prevent inflammation, cartilage degradation, and post traumatic osteoarthritis.

The “site of inflammation” refers to the specific tissue or area in the subject's body that exhibits inflammation. Inflammation can be caused by many factors, including physical trauma (including burns, freezing, foreign bodies, degeneration from use or overuse, and the like), infection, cancer, chemical exposure (including exposure to smoke), radiation, ischemia, auto-immune disorders, asthma, and the like. Similarly, the “site of traumatic injury” refers to that part of the subject's body that has experienced a trauma. The site of traumatic injury can be soft tissue or hard tissue (for example, bones, cartilage, and joints).

### III. Compositions and Methods

The methods and compositions herein provide sustained-release formulations of a CDK9 inhibitor for local delivery. One important advantage of encapsulating the active drug in a sustained release formulation is that the drug remains locally available at therapeutically effective concentrations over an extended period of time. The duration of the release is moderated by parameters such as the inherent viscosity of the polymer, L:G ratio, the termination group of the polymer and particle size. As shown herein, these parameters can be engineered to correspond to the duration of the typical inflammatory response, which can range from days to weeks after an acute injury event. This is an improvement over conventional systemic administration of the drug, as it would be quickly metabolized and inactivated (for example, flavopiridol has an in-vivo half-life of about 5-6 hours). A second important advantage of the formulations of the invention is the local delivery of the drug. For example, when microparticles with encapsulated flavopiridol are injected intra-articularly, they remain within the joint capsule This provides a therapeutically effective local concentration of the drug within the joint space over time, while greatly reducing the systemic drug burden.

A. Compounds

Provided herein are formulations of therapeutic agents that target Cdk9 kinase activity using existing small-molecule inhibitors of CDK9. The formulations provided herein are suitable with flavopiridol, voruciclib and the class of CDK9 inhibitors structurally related to flavopiridol and voruciclib such that the inhibitor is delivered with appropriate overall release potential and release kinetics to the affected site of a subject, such as an injured tissue or cell type.

In some embodiments, the CDK9 inhibitor is flavopiridol, or an ester, prodrug, or pharmaceutically acceptable salt thereof. In some embodiments, the CDK9 inhibitor is flavopiridol, or derivative or salt thereof. In some embodiments, the CDK9 inhibitor is flavopiridol, SNS-032, or voruciclib.

In some embodiments, the CDK9 inhibitor is flavopiridol, or an ester, prodrug, or pharmaceutically acceptable salt thereof. In some embodiments, the CDK9 inhibitor is flavopiridol, or a derivative or salt thereof. In some embodiments, the CDK9 inhibitor is flavopiridol (IUPAC name: 2-(2-chlorophenyl)-5,7-dihydroxy-8-[(3S,4R)-3-hydroxy-1-methyl-4-piperidinyl]-4-chromenone; CAS #146426-40-6), having a structure of:

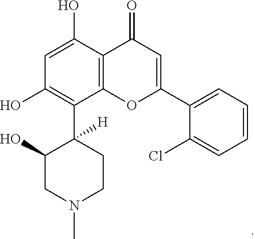 [SMILES: CN1CC[C@H](c2c(O)cc(O)c3c(=O)cc(-c4ccccc4Cl)oc23)[C@H](O)C1]

CDK9 inhibitors such as flavopiridol broadly and efficiently suppress the transcriptional activation of primary response genes, which includes inflammatory genes (such as IL-1, TNF, IL-6, iNOS, etc.) and matrix degrading enzymes (MMPs, ADAMTS, etc.). However, flavopiridol is rapidly metabolized and degraded, and has a short in-vivo half-life of under 6 hours. As a small molecule (˜400 Da), it rapidly diffuses from the site of administration, and is therefore generally given as a systemic administration. Provided herein are formulations of a CDK9 inhibitor and a PLGA polymer, wherein the CDK9 inhibitor is encapsulated in a particle of appropriate size and with appropriate release potential and release kinetics such that the CDK9 inhibitor is provided in a therapeutically effective amount over a duration to treat an injury, reduce inflammation, ameliorate symptoms and/or prevent further damage to an injured tissue of a subject.

In some embodiments, the CDK9 inhibitor is SNS-032, or a prodrug, or a pharmaceutically acceptable salt thereof. In some embodiments, the CDK9 inhibitor is SNS-032, or a salt thereof. In some embodiments, the CDK9 inhibitor is SNS-032, having the structure:

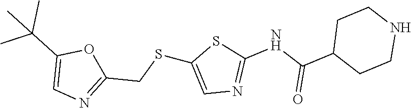 [SMILES: CC(C)(C)c1cnc(CSc2cnc(NC(=O)C3CCNCC3)s2)o1]

In some embodiments, the CDK9 inhibitor is voruciclib, or an ester, prodrug, or pharmaceutically acceptable salt thereof. In some embodiments, the CDK9 inhibitor is voruciclib, or a derivative or salt thereof. In some embodiments, the CDK9 inhibitor is voruciclib, having the structure:

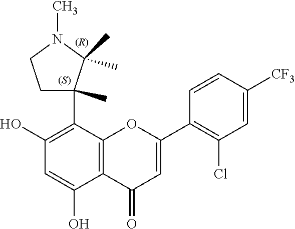

Another CDK9 inhibitor is dinaciclib. Dinaciclib is not encapsulated effectively or released appropriately in microparticles of the invention:

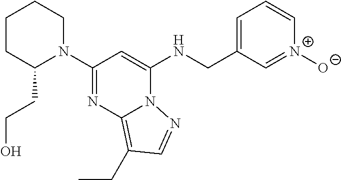 [SMILES: CCc1cnn2c(NCc3ccc[n+]([O-])c3)cc(N3CCCC[C@H]3CCO)nc12]

Provided herein are formulations of a CDK9 inhibitor wherein the CDK9 inhibitor is SNS-32, voruciclib, or flavopiridol, and a PLGA polymer such that the CDK9 inhibitor, is encapsulated in a microparticle of appropriate size and with appropriate release potential and release kinetics such that the CDK9 inhibitor is provided in a therapeutically effective amount over a duration to treat an injury, reduce inflammation, ameliorate symptoms and/or prevent further damage to an injured tissue of a subject.

Provided are also pharmaceutically acceptable salts, hydrates, solvates, tautomeric forms, polymorphs, and prodrugs of the CDK9 inhibitors described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts, or when appropriate as a free base. “Pharmaceutically acceptable salts” are non-toxic salts of a free base form of a compound that retain the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Other suitable pharmaceutically acceptable salts are found in Remington: The Science and Practice of Pharmacy, 21^{st} Edition, Lippincott Williams and Wilkins, Philadelphia, Pa., 2006.

Examples of pharmaceutically acceptable salts of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

B. Compositions

Provided herein are novel sustained-release formulations of a CDK9 inhibitor in which the inhibitor is encapsulated in a bioresorbable polymer. The inhibitor is continuously released over time as the polymer degrades. The polymer is in the form of microparticles, which are retained at the site of administration (for example, intra-articular injection to an injured joint). In some embodiments, the microparticles range in size from 4 microns to 50 microns in diameter, with a target size of approximately 15 microns in diameter. In some embodiments the average diameter of the microparticles is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 microns in diameter. In some embodiments, the average diameter of the microparticles is less than or equal to 100 microns, less than or equal to 70 microns, less than or equal to 50 microns, less than or equal to 45 microns, less than or equal to 40 microns, less than or equal to 35 microns less than or equal to 30 microns less than or equal to 25 microns less than or equal to 20 microns less than or equal to 15 microns, less than or equal to 10 microns, or less than or equal to 5 microns. In some embodiments, the average diameter of the microparticles is between about 20 microns to about 50 microns, between about 20 microns to about 30 microns or between about 10 microns to about 20 microns. In some embodiments, the average diameter of the microparticles is between about 12 microns to about 18 microns. In some embodiments, the average diameter of the microparticles is about 15 microns, about 20 microns, about 25 microns about 30 microns about 35 microns about 40 microns, about 45 microns or about 50 microns. In some embodiments, the bioresorbable polymer is PLGA. The microparticles are retained at the site of administration (for example, intra-articular injection to an injured joint). The advantage microparticle-encapsulated drug formulation is that the microparticles stay localized where injected, thus the inhibitor is locally available at therapeutically effective concentrations over a prolonged period of time that can be engineered precisely. The timing, for example, can be engineered to release the drug in the time span of the catabolic inflammatory phase of injury response, from days to months.

An embodiment of the invention is a microparticle comprising a cyclin-dependent kinase 9 (CDK9) inhibitor and poly(lactic-co-glycolic) acid (PLGA), wherein the CDK9 inhibitor is encapsulated by the PLGA, and wherein the microparticle provides a sustained release of the CDK9 inhibitor.

In some embodiments, the bioresorbable polymer is a PLGA copolymer. PLGA copolymers that are useful for sustained release of the CDK9 inhibitor of the disclosure include those that degrade at a rate such that the CDK9 inhibitor is substantially released over the course of about 30 days. In some embodiments, PLGA copolymers include those that comprise from about 10:90 to about 90:10 ratio of lactic acid to glycolic acid monomers (L:G ratio). In some embodiments, PLGA copolymers include those that comprise from about 50:50 to about 75:25 ratio of lactic acid to glycolic acid monomers (L:G ratio), including copolymers having about 50:50 to about 75:25. In some embodiments, PLGA copolymers include those that comprise an L:G ratio of about 70:30, about 65:35, about 60:40, about 60:50, or about 55:45. In some embodiments herein the PLGA copolymer of the disclosure is acid terminated. In embodiments of the invention, the PLGA is an acid-terminated 50:50 poly(DL-lactide-co-glycolide). In embodiments of the invention, the PLGA is a Lactel® polymer (Durect Corp.). In embodiments of the invention, the PLGA is a Lactel® B6013-1, B6013-2, or B6012-4 polymer. In embodiments of the invention, the PLGA is a Purasorb® polymer (Corbion). In embodiments of the invention, the PLGA is a Purasorb® 5004A 50:50 poly(DL-lactide-co-glycolide).

In some embodiments, the PLGA-encapsulated CDK9 inhibitor microparticles are from about 1 to about 50 microns in diameter, e.g., from about 1 to about 50, about 1 to about 40, about 2 to about 50, about 2 to about 40, about 3 to about 50, or from about 3 to about 40 microns in diameter. Uniform production of micron-sized particles is desired for use in a method of the invention, with at least about 90%, 95%, 96%, 98% or at least about 99% of the mass of the particles for use in a pharmaceutical formulation having a diameter of less than about 20, about 25, about 30, about 35, about 40, about 45, about 50, about 60, about 70, about 80, about 90 or about 100 microns.

In some embodiments of the invention, the microparticles release the CDK9 inhibitor at approximately a constant rate over the treatment period. In some embodiments, the microparticles release at a constant rate after an initial release of about 3% to about 10% of the encapsulated CDK9 inhibitor. In some embodiments, the initial release occurs within about 24 hours. In some embodiments, the initial release occurs within about 12 hours. In some embodiments, the initial release occurs within about 8 hours. In some embodiments, the initial release occurs within about 1 hour. In some embodiments, the microparticle releases from about 3% to about 30%, about 3% to about 20%, about 3% to about 10%, about 5% to about 30%, about 5% to about 20%, or from about 5% to about 10% of the CDK9 inhibitor over 24 hours. In some embodiments, the microparticle releases from about 5% to about 40%, about 5% to about 30%, about 5% to about 20%, about 10% to about 40%, about 10% to about 30%, about 10% to about 20%, or from about 10% to about 15% of the CDK9 inhibitor over 2 days. In some embodiments, the microparticle releases from about 10% to about 50%, about 10% to about 40%, about 10% to about 30%, about 15% to about 40%, about 15% to about 30%, or from about 15% to about 25% of the CDK9 inhibitor over 5 days. In some embodiments, the microparticle releases from about 20% to about 70%, about 20% to about 60%, about 20% to about 50%, about 20% to about 40%, or from about 25% to about 35% of the CDK9 inhibitor over 8 days. In some embodiments, the microparticle releases from about 30% to about 70%, about 30% to about 60%, about 30% to about 50%, about 40% to about 70%, about 40% to about 60%, or from about 40% to about 50% of the CDK9 inhibitor over 12 days. In some embodiments, the microparticle releases from about 40% to about 80%, about 40% to about 70%, about 50% to about 70%, or from about 55% to about 65% of the CDK9 inhibitor over 15 days. In some embodiments, the microparticle releases from about 40% to about 80%, about 50% to about 80%, about 60% to about 80%, or from about 65% to about 75% of the CDK9 inhibitor over 19 days. In some embodiments, the microparticle releases from about 40% to about 90%, about 50% to about 90%, about 60% to about 90%, about 70% to about 90%, or from about 75% to about 85% of the CDK9 inhibitor over 22 days. In some embodiments, the microparticle releases from about 50% to about 90%, about 60% to about 90%, about 70% to about 90%, or from about 80% to about 90% of the CDK9 inhibitor over 26 days. In some embodiments, the microparticle releases from about 60% to about 95%, about 70% to about 95%, about 80% to about 95%, or from about 85% to about 95% of the CDK9 inhibitor over 30 days.

In embodiments of the invention, at least about 80% of the encapsulated CDK9 inhibitor is released by the end of the treatment period. In some embodiments, the amount of encapsulated CDK9 inhibitor released is at least about 85%, at least about 90%, at least about 94%, at least about 95%, at least about 96%, at least about 97%, at least about 98%, at least about 99%, or at least about 99.5%. In embodiments of the invention, the treatment period is at least about 24 hours, at least about 2 days, at least about 5 days, at least about 7 days, at least about 10 days, at least about 14 days, at least about 20 days, at least about 21 days, at least about 28 days, at least about 30 days, at least about 31 days, at least about 40 days, at least about 42 days, at least about 45 days, at least about 48 days, at least about 50 days, or at least about 60 days. In embodiments of the invention, the treatment period is less than about 60 days, less than about 55 days, less than about 50 days, less than about 45 days, less than about 40 days, less than about 30 days, less than about 28 days, less than about 25 days, less than about 21 days, less than about 20 days, less than about 14 days, less than about 10 days, less than about 7 days, less than about 5 days, or less than about 2 days.

In some embodiments, the microparticle releases from about 3% to about 10% of the CDK9 inhibitor over about 24 hours; from about 10% to about 20% of the CDK9 inhibitor over about 2 days; from about 15% to about 25% of the CDK9 inhibitor over about 5 days; from about 25% to about 35% of the CDK9 inhibitor over about 8 days; from about 40% to about 50% of the CDK9 inhibitor over about 12 days; from about 55% to about 65% of the CDK9 inhibitor over about 15 days; from about 65% to about 75% of the CDK9 inhibitor over about 19 days; from about 75% to about 85% of the CDK9 inhibitor over about 22 days; from about 80% to about 90% of the CDK9 inhibitor over about 26 days; and/or from about 85% to about 95% of the CDK9 inhibitor over about 30 days.

An embodiment of the invention is a microparticle wherein the CDK9 inhibitor is flavopiridol, SNS-032, voruciclib, or a pharmaceutically acceptable salt thereof. An embodiment of the invention is a microparticle wherein the CDK9 inhibitor is flavopiridol. An embodiment of the invention is a microparticle wherein the PLGA has a lactic acid to glycolic acid (L:G) ratio of about 50:50 to about 75:25. An embodiment of the invention is a microparticle wherein the PLGA has an inherent viscosity (IV) of from about 0.4 to about 0.9. An embodiment of the invention is a microparticle wherein the PLGA has an inherent viscosity (IV) of about 0.4, about 0.55 to about 0.75, or about 0.7 to about 0.9. An embodiment of the invention is a microparticle wherein the PLGA is Lactel® B6013-2, Purasorb® 5004A, or Lactel® B6012-4. An embodiment of the invention is a microparticle wherein the microparticle has a diameter of from about 3 to about 50 microns.

An embodiment of the invention is a microparticle wherein the microparticle releases the CDK9 inhibitor over a duration selected from the group consisting of about 24 hours, about 2 days, about 5 days, about 10 days, about 14 days, about 21 days, about 30 days, about 45 days, and about 60 days. An embodiment of the invention is a microparticle wherein the microparticle releases from about 5% to about 40%, about 5% to about 30%, about 5% to about 20%, about 10% to about 40%, about 10% to about 30%, about 10% to about 20%, or from about 10% to about 15% of the CDK9 inhibitor over 2 days following administration. An embodiment of the invention is a microparticle wherein the microparticle releases from about 10% to about 50%, about 10% to about 40%, about 10% to about 30%, about 15% to about 40%, about 15% to about 30%, or from about 15% to about 25% of the CDK9 inhibitor over 5 days following administration. An embodiment of the invention is a microparticle wherein the microparticle releases from about 20% to about 70%, about 20% to about 60%, about 20% to about 50%, about 20% to about 40%, or from about 25% to about 35% of the CDK9 inhibitor over 8 days following administration. An embodiment of the invention is a microparticle wherein the microparticle releases from about 30% to about 70%, about 30% to about 60%, about 30% to about 50%, about 40% to about 70%, about 40% to about 60%, or from about 40% to about 50% of the CDK9 inhibitor over 12 days following administration. An embodiment of the invention is a microparticle wherein the microparticle releases from about 40% to about 80%, about 40% to about 70%, about 50% to about 70%, or from about 55% to about 65% of the CDK9 inhibitor over 15 days following administration. An embodiment of the invention is a microparticle wherein the microparticle releases from about 40% to about 80%, about 50% to about 80%, about 60% to about 80%, or from about 65% to about 75% of the CDK9 inhibitor over 19 days following administration. An embodiment of the invention is a microparticle wherein the microparticle releases from about 40% to about 90%, about 50% to about 90%, about 60% to about 90%, about 70% to about 90%, or from about 75% to about 85% of the CDK9 inhibitor over 22 days following administration. An embodiment of the invention is a microparticle wherein the microparticle releases from about 50% to about 90%, about 60% to about 90%, about 70% to about 90%, or from about 80% to about 90% of the CDK9 inhibitor over 26 days following administration. An embodiment of the invention is a microparticle wherein the microparticle releases from about 60% to about 95%, about 70% to about 95%, about 80% to about 95%, or from about 85% to about 95% of the CDK9 inhibitor over 30 days following administration.

Another embodiment of the invention is a pharmaceutical composition comprising a plurality of microparticles of the invention and a pharmaceutically acceptable carrier. An embodiment of the invention is the composition wherein the plurality of microparticles has a mean diameter of from about 5 to about 20, or from about 10 to about 20 microns, or from about 20 to about 50 microns. An embodiment of the invention is the composition wherein 10% of the mass of the plurality of microparticles (D10) has a diameter of less than about 9 or about 10 microns. An embodiment of the invention is the composition wherein 50% of the mass of the plurality of microparticles (D50) has a diameter of less than about 18, less than about 19, or less than about 20 microns. An embodiment of the invention is the composition wherein 90% of the mass of the plurality of microparticles (D90) has a diameter of less than about 26, about 27, about 28, about 29, or about 30 microns. An embodiment of the invention is the composition wherein the plurality of microparticles has from about 0.5% to about 5%, about 0.5% to about 4%, about 0.5% to about 3%, or from about 0.5% to about 2% by weight of the CDK9 inhibitor.

Pharmaceutical compositions of the invention comprise microparticles of the invention dispersed or suspended in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, dyes, like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the microparticles of the invention, its use in the pharmaceutical compositions is contemplated. It is anticipated that the compositions of the invention will be administered primarily by injection or other parenteral methods; however, gel and aerosol compositions may also be used, for example, for application during a surgical procedure. Suitable carriers include water, water for injection, saline, phosphate buffered saline, and the like. Compositions of the invention can further include propellants, anti-aggregation agents, and the additional agents listed above.

An embodiment of the invention is a method wherein the microparticles are administered in a pharmaceutically acceptable carrier. An embodiment of the invention is a method wherein the CDK9 inhibitor is selected from the group consisting of flavopiridol, SNS-032, voruciclib, and a derivative thereof, or pharmaceutically acceptable salt thereof. An embodiment of the invention is a method wherein the CDK9 inhibitor is flavopiridol, SNS-032, or voruciclib, or a pharmaceutically acceptable salt thereof. An embodiment of the invention is a method wherein the CDK9 inhibitor is flavopiridol. An embodiment of the invention is a method wherein the subject treated is a human. An embodiment of the invention is a method wherein the subject treated is an equine. An embodiment of the invention is a method wherein a therapeutically effective amount of the CDK9 inhibitor is released over a duration of 1 to 42 days.

In an embodiment of the invention, the composition comprises a carrier that comprises water and polyvinyl alcohol (PVA). In an embodiment of the invention, the carrier comprises ethanol, a polyol (i.e., glycerol, propylene glycol, or liquid polyethylene glycol, and the like), or a suitable mixture thereof. In an embodiment of the invention, the carrier comprises a gelling agent. In compositions of the invention that are to be hydrated or suspended immediately prior to administration, the carrier may be a dry particulate solid suitable for suspending and disaggregating the microparticles of the invention, for example mannitol, sucrose, and the like.

C. Methods

Another embodiment of the invention is a method of treating a subject in need thereof, comprising administering a therapeutically effective amount of a plurality of microparticles, the microparticles comprising a CDK9 inhibitor and a poly(lactic-co-glycolic) acid (PLGA), wherein the CDK9 inhibitor is encapsulated by the PLGA, and wherein the microparticles provide a sustained release of the CDK9 inhibitor.

The methods herein provide formulated sustained-release CDK9 inhibitors, for local delivery such that the drug remains locally available at therapeutically effective doses over an extended period of time. The CDK9 inhibitor formulated with a formulating agent into microparticles provides release of the CDK9 inhibitor over the duration of an inflammatory response, which can range from days to weeks after an acute injury event. Also provided herein are methods of administering formulated CDK9 inhibitors for local delivery of the drug. For example, microparticles with encapsulated CDK9 inhibitors remain within tissue of interest, such as a joint capsule when injected intra-articularly to provide a therapeutically effective local concentration of the drug within the tissue over time, while greatly reducing the drug burden systemically.

The subject that can be treated with a method of the present disclosure is a human, or a non-human mammal, for example a companion animal, such as a dog, cat, rat, or the like, or a farm animal, such as a horse, donkey, mule, goat, sheep, pig, or cow, or the like.

The systemic drug burden is greatly reduced in the method of the invention, as the therapeutic dose is administered locally, and thus a much lower dose can be used. For example, when administering a composition of the invention by intra-articular injection of a single knee joint, a locally effective concentration of the CDK9 inhibitor, such as flavopiridol can be achieved with approximately 80- to 100-fold less drug than a systemic dose in humans, and an even greater reduction in the case of an injured equine joint. A sustained release approach is useful in order to significantly reduce complications associated with post-traumatic systemic and local hyperinflammation.

These complications that are avoided can include acute lung injury, fat embolism, multiple organ failure, delay healing, severe post-injury immunosuppression etc. This invention can be used to reduce inflammation-induced swelling, limit tissue damage in severe brain/spinal cord trauma, prevent systemic inflammation in severe multifocal trauma cases such as those received in automobile accidents, limit muscle damage after myocardial infarction, and other conditions in which the acute inflammatory response is undesirable. The invention is particularly suited to situations where a secondary immune response causes undesired effects. Specific examples include: (a) joint injury such as meniscal tear or ACL tear, where the immune response activates cartilage matrix degrading enzymes that predispose the joint to future osteoarthritis, (b) neurological damage from toxins (nerve gas, organophosphates, etc.) where the immune response can be pro-convulsant, (c) medical implants where a local immune response or foreign-body response is not desired.

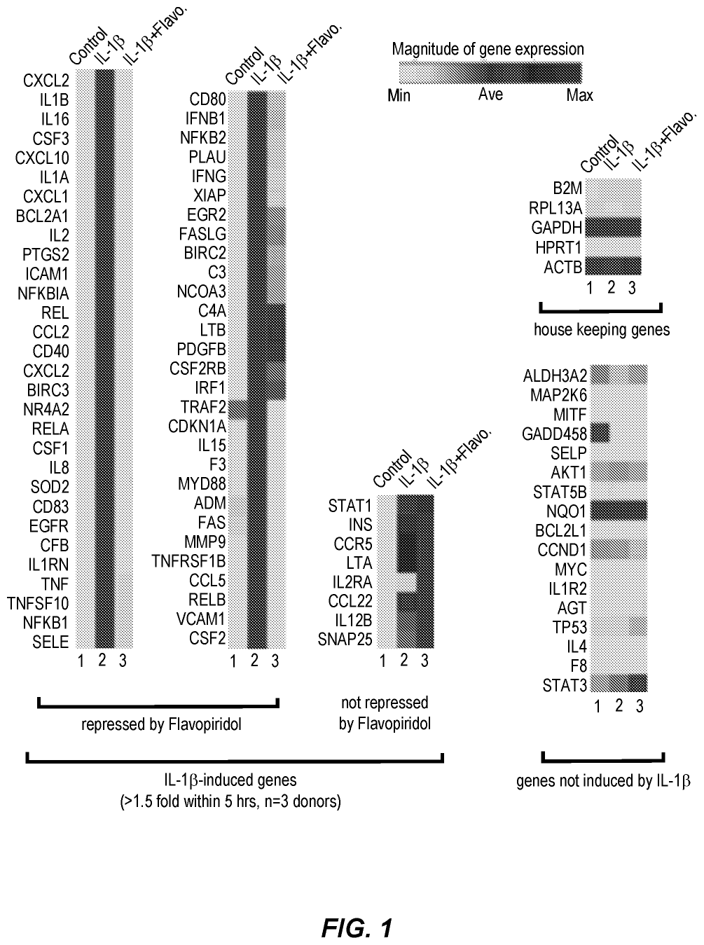

CDK9 inhibitors exert effects on the inflammatory response pathway. For example, the pharmacological CDK9 inhibitor flavopiridol effectively suppresses the activation of a broad range of primary inflammatory response genes, in human cell culture treated with IL-1β for 5 hours (see FIG. 1). Among the 67 different genes (out of 84 total NFκB target genes tested) that were induced by IL-1β, 59 were repressed by flavopiridol co-treatment (including the most-characterized pro-inflammatory cytokines such as IL-1β, Il-6, and TNF). The average magnitude of repression is >86% of maximum induction. These data demonstrate that CDK9 inhibition is highly efficient in suppressing the induction of a broad range of primary inflammatory genes. Importantly, house-keeping genes and non-inducible genes are not affected by CDK9 inhibition short term, indicating potential reduction in side effects.

Current anti-inflammatory drugs either target various components of the upstream inflammatory signaling pathways, or the downstream effector genes (IL-1 antagonists, TNF antagonists, anti-oxidants, etc.). The focus has been on inhibition of the specific pathway(s) so that transcription of corresponding response genes does not occur, or on inhibition of individual downstream effector gene functions. None of these existing investigations have addressed the rate-limiting process of transcriptional elongation that is controlled by CDK9. These existing drugs may be less effective in handling the diverse physiological pro-inflammatory challenges, and may not be able to prevent activation of a broad range of different downstream inflammatory response genes. Therefore, targeting CDK9 that controls the rate-limiting step for all inflammatory gene activation is more effective and efficient. Inhibition of the transcriptional elongation by CDK9 is limited to the primary response inflammatory genes, and CDK9 inhibition does not affect transcription of housekeeping genes and non-inducible genes within the acute inflammatory phase tested, and therefore is not detrimental to cells or tissues in the short term. One advantage of CDK9 inhibition is that it reduces transcriptional elongation of inflammatory genes from numerous inflammatory stimuli. CDK9 can be specifically and reversibly inhibited with small-molecule drugs such as flavopiridol and others disclosed herein, including SNS-032, voruciclib, and flavopiridol. In conjunction with the formulations and methods herein, CDK9 inhibitors are delivered locally to a site of inflammation and thereby reduce, alleviate, prevent or reduce inflammatory response and symptoms thereof.

In some embodiments, the methods herein include administering at least one CDK9 inhibitor and a PLGA polymer in the form of microparticles described herein, wherein the CDK9 inhibitor is selected from the group consisting of flavopiridol, SNS-032, and voruciclib, or an ester, prodrug, or pharmaceutically acceptable salt thereof.

In some embodiments, the formulated CDK9 inhibitor is administered to a target tissue, cell type or region of a subject's body, including but not limited to an injured site, an area of inflammation or potential inflammation, a joint, cartilage, a tissue that has experienced a surgery, a tissue or area damaged by a sports injury, an explant such as an osteochondral explant, including but not limited to allograft cartilage, an area of cartilage degradation and/or chondrocyte death.

In some embodiments, the formulated CDK9 inhibitor is administered within 10 days of a traumatic injury or inflammation response. In some embodiments, the formulated CDK9 inhibitor is administered within 10, 9, 8, 7, 6, 5, 4, 3, 2 or 1 days after a traumatic injury or inflammation response. In some embodiments, the formulated CDK9 inhibitor is administered within 24, 23, 22, 21, 20, 19, 18, 17, 16, 15, 14, 13, 12, 11, 10, 9, 8, 7, 6, 5, 4, 3, 2, 1, 0.5 hours or less than 0.5 hours after a traumatic injury or inflammation response. In some embodiments, the formulated CDK9 inhibitor is administered once, twice, 3 times, or more after a traumatic injury or inflammation response.

In some embodiments, the formulated CDK9 inhibitor is administered to a subject having a pre-existing condition or disease such as synovitis or arthritis. In some embodiments, the formulated CDK9 inhibitor is administered for such pre-existing condition on a chronic basis, such as every week, every 2 weeks, every 3 weeks, every month (e.g. 4 weeks), every 5, 6, 7, 8, 9 or 10 weeks. In some embodiments, the formulated CDK9 inhibitor is administered for such pre-existing condition on a chronic basis, until the symptoms, inflammation or other signs of the condition or disease are reduced, ameliorated, dampened or otherwise effected by the treatment. In some embodiments, the formulated CDK9 inhibitor is administered for such pre-existing condition on a chronic basis for the life-time of a subject or from the time of diagnosis or flare-up of the disease or condition.

Another embodiment of the invention is a method of treating a subject in need thereof, comprising administering a pharmaceutical composition comprising a plurality of microparticles of the invention.

An embodiment of the invention is a method wherein the subject has a disease or condition selected from arthritis, osteoarthritis, post-traumatic osteoarthritis, and a traumatic injury. An embodiment of the invention is a method wherein the disease or condition effects an articular joint. An embodiment of the invention is a method wherein the articular joint is a knee joint. An embodiment of the invention is a method wherein the pharmaceutical composition is administered by injection.

Another embodiment of the invention is a method of treating a site of inflammation comprising, administering to the site a composition comprising a CDK9 inhibitor formulated into a plurality of microparticles, wherein the microparticles provide a sustained release of the CDK9 inhibitor at the site for at least 24 hours, and whereby inflammation at the site is thereby reduced or ameliorated.

An embodiment of the invention is a method wherein the site of inflammation is a joint, cartilage, or a site of traumatic injury. An embodiment of the invention is a method wherein the microparticles comprise PLGA, and wherein the CDK9 inhibitor is selected from the group consisting of flavopiridol, SNS-032, voruciclib, and a derivative thereof, or a pharmaceutically acceptable salt thereof. An embodiment of the invention is a method wherein the microparticles have an average diameter between about 20 to about 50 microns.

### IV. Examples

### Example 1. Synthesis of Particles Comprising Poly(lactic-co-glycolic) Acid and Flavopiridol

Preparation of flavopiridol-poly(lactic-co-glycolic) acid (PLGA) particles was performed using a single emulsion-solvent evaporation technique. Briefly, PLGA was dissolved in methylene chloride (5% w/v), flavopiridol added, and the solution added to a bulk volume of a polyvinyl alcohol in distilled water while homogenizing (35,000 rpm for 2 min) to form an emulsion. Particles, thus formed, were stirred for 24 h to evaporate residual methylene chloride. MPs were washed, lyophilized and stored at −20° C. Size distribution was measured by a Microtrac Nanotrac Dynamic Light Scattering Particle Analyzer, and confirmed by scanning electron microscopy.

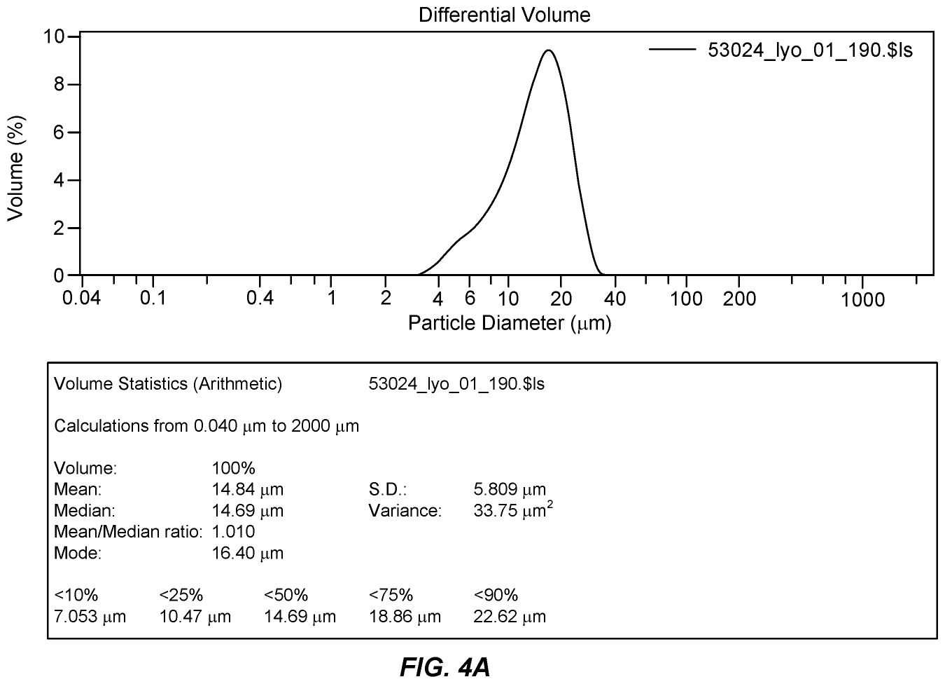

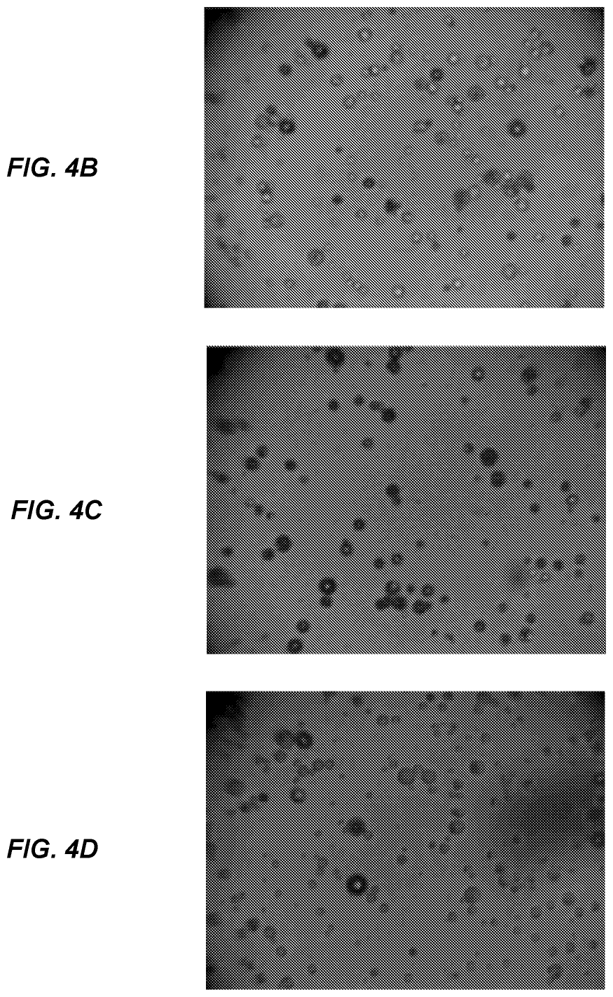

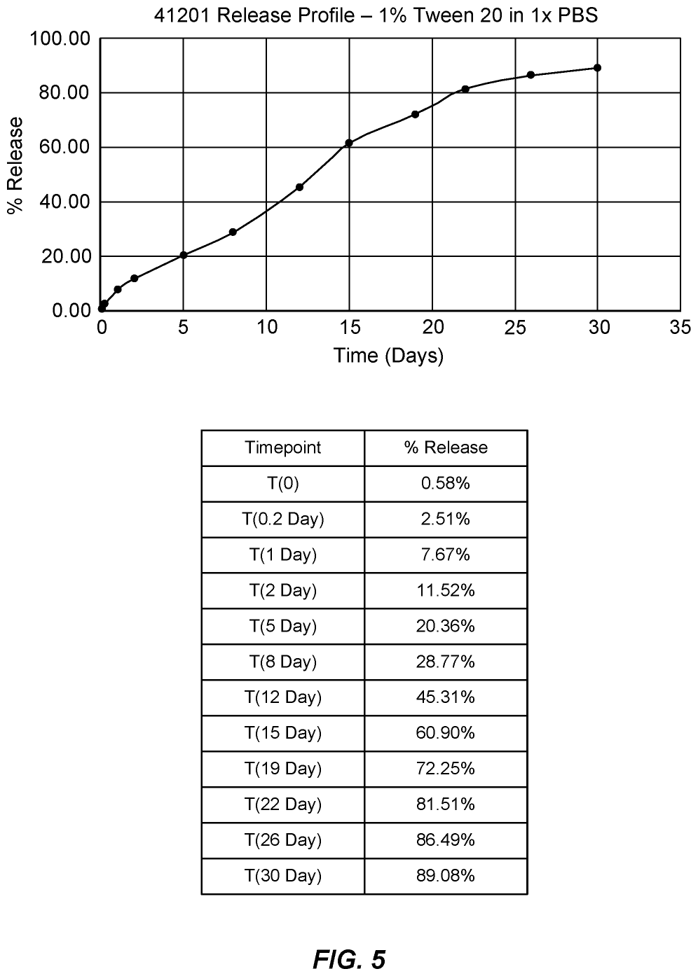

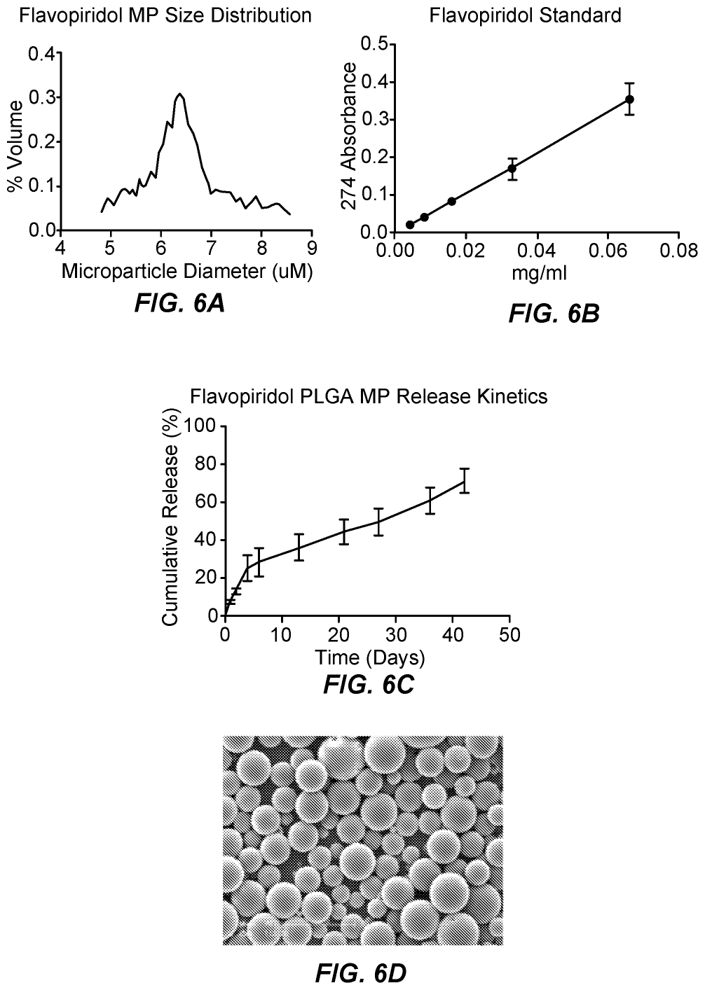

We have produced different versions of CDK9-inhibitor-releasing microparticles that show sustained release of flavopiridol. Examples are shown in Table 1 and FIG. 4A-D and FIG. 5. The differences in these formulations stem from the characteristics of the PLGA that encapsulates the CDK9 inhibitor (in this case flavopiridol), which in turn affects the kinetics of flavopiridol release. These polymers were chosen based on their physical properties, such as inherent viscosity or average molecular weight, their compatibility with CDK9 inhibitors, such as flavopiridol, their IG ratio, and their predicted release kinetics. In this case, the polymers were Lactel® B6013-2, Purasorb® 5004A (Corbion), and Lactel® B6012-4.

The characteristics of these polymers are shown in Table 1. The microparticles are of a median size of approximately 15 microns (range 4-50 microns), with a flavopiridol content of approximately 0.5% to 1.5% by weight (FIG. 4).

TABLE 1
Formulations of PLGA-encapsulated flavopiridol particles with
given properties (L/G ratio, inherent viscosity, termination group).
Formulation 1: Lactel ® B6013-2, LG ratio 50:50, IV:
0.55-0.75 dL/g, acid terminated; Formulation 2: Purasorb ®
5004A, LG ratio 50:50, IV 0.4 dL/g, acid terminated; Formulation
3: Lactel ® B6012-4, LG ratio 75:25, IV 0.7-0.9 dL/g, acid
terminated.

| | Formulation 1 | Formulation 2 | Formulation 3 |
|---|---|---|---|
| Lot Number | 53010 | 53012 | 53024 |
| Ave. Particle Size | 16.21 ± 6.229 μm | 16.72 ± 60.35 μm | 14.84 ± 5.809 μm |
| D10 | 8.007 μm | 8.528 μm | 7.053 μm |
| D50 | 16.08 μm | 16.79 μm | 14.69 μm |
| D90 | 24.95 μm | 25.01 μm | 22.62 μm |
| API loading | 1.58% | 1.13% | 0.51% |

### Example 2. In Vitro Evaluation of PLGA/Flavopiridol Particles

Flavopiridol release from the particles was quantified over 42 days in PBS-Tween®, by absorbance at 247 nm. FIG. 5 shows nearly linear release kinetics out to 30 days from one of the polymers, with approximately 90% of the flavopiridol released from the microparticles by 30 days in-vitro.

### Example 3. PLGA/Flavopiridol Particles are Active in Rat Osteoarthritis Model

To induce post-traumatic osteoarthritis (PTOA) in rats (IACUC approved), 4 rats (Sprague Dawley) were anesthetized and a single mechanical overload applied to the knee joint to rupture the ACL. 5 mg of particles were suspended in 50 μl saline and administered into the intra-articular space using a 23-gauge needle, with 2 rats receiving flavopiridol-PLGA and 2 rats receiving blank-PLGA. To assess OA development and joint degradation, we performed longitudinal (up to 3 weeks) in-vivo imaging of MMP activity using intraarticular injections of MMPSense750-FAST on an IVIS-200.

In the rat PTOA model, we observed a strong increase in the in-vivo MMP activity 3 days after injury. However, intra-articular injection of flavopiridol-PLGA microparticles markedly reduced the in-vivo MMP activity at all time points tested.

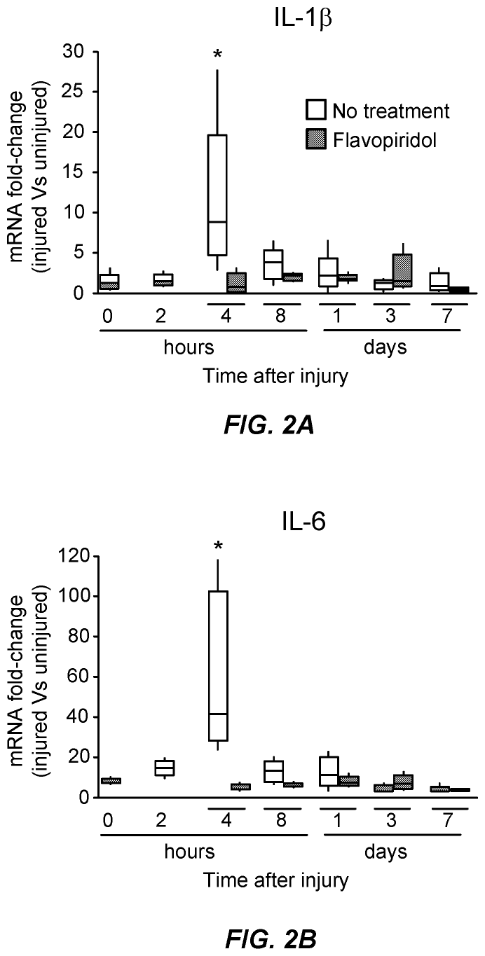

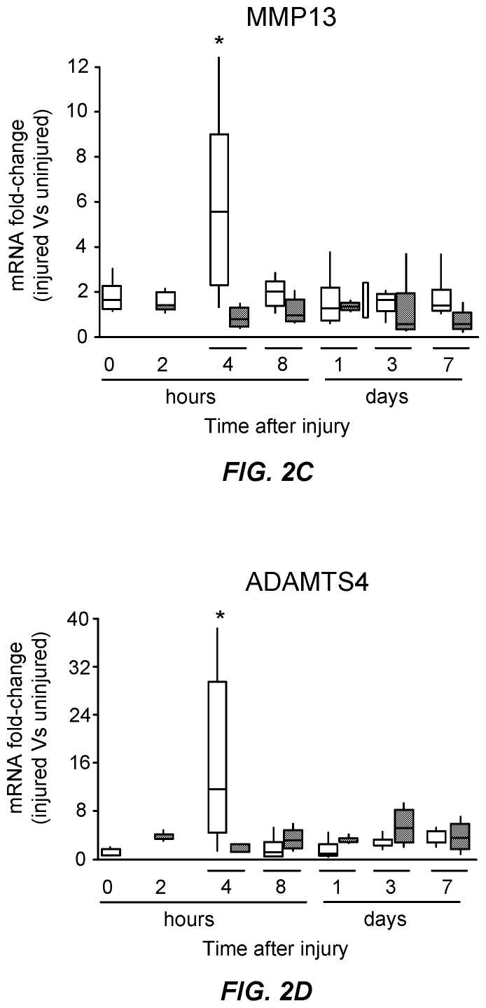

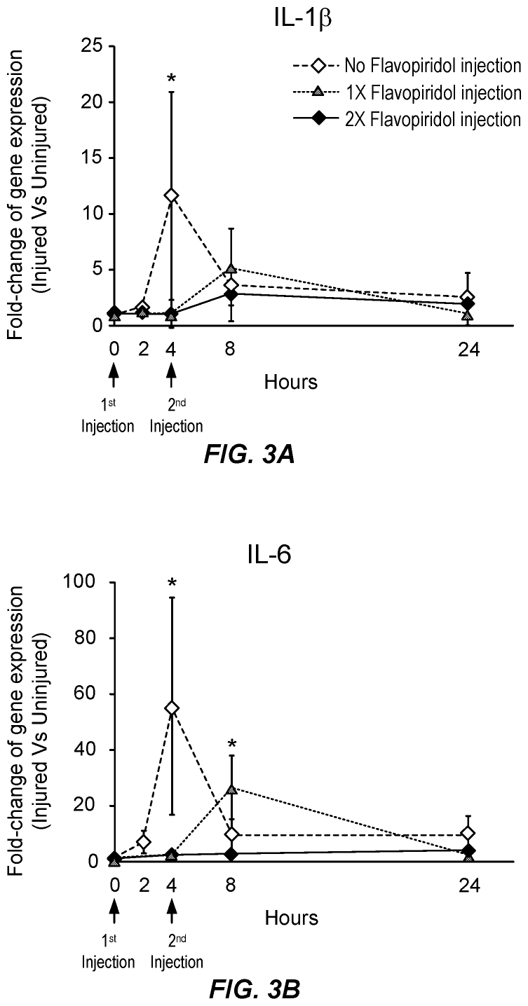

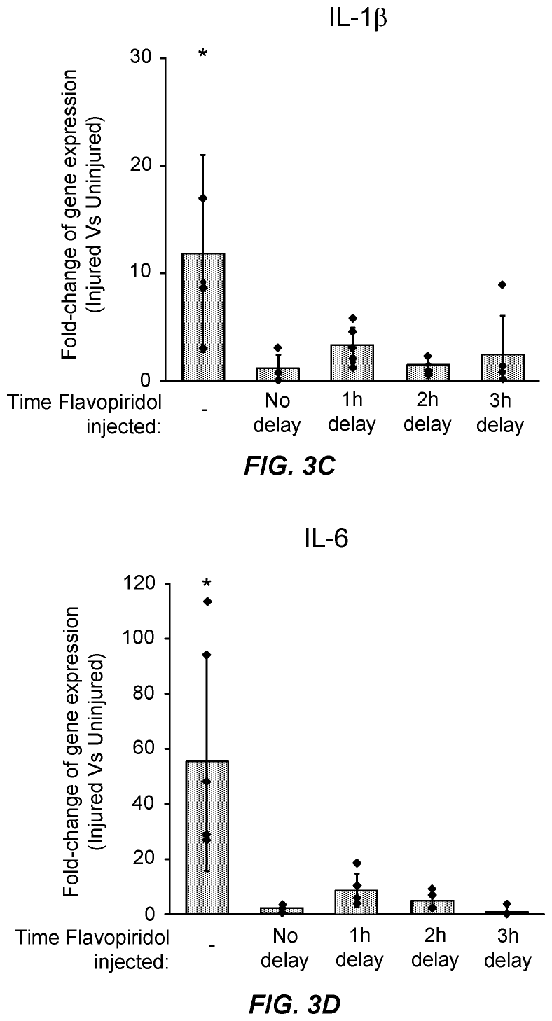

Moreover, in vivo data in the context of joint injury showed that flavopiridol injection effectively and selectively suppressed the mRNA expression of pro-inflammatory cytokines IL-1β and IL-6 at the injured site 4-8 hours post-injury (see FIG. 2A-2D, FIG. 3A-3D), as assessed by a non-invasive knee injury model that we developed for studying post-traumatic osteoarthritis (B. A. Christiansen et al., Osteoarthritis Cartilage (2012) 20(7):773-82). In addition, the house-keeping gene 18S rRNA, as well as matrix gene Collagen Type 2 and aggrecan were not affected by the injury nor flavopiridol treatment. These data indicate that CDK9 inhibition is effective in suppressing the production of the major pro-inflammatory cytokines IL-1β and IL-6 in vivo at the site of injury, even though flavopiridol was administered distally and systemically through intraperitoneal injection. The data in FIG. 3A and FIG. 3B show that repeated administration of flavopiridol is more effective than a single administration.

Additional in-vivo data was generated in a rat model where PTOA is initiated by ACL-Rupture similar to the mouse study referenced above. In this example, PLGA-encapsulated CDK9 inhibitor flavopiridol was delivered by intra-articular injection to the injured knee joint. There was no apparent toxicity or other negative reaction to the formulation. The formulation had a potent effect in reducing MMP activity in ACL-rupture joints. The injury-induced activation of catabolic enzymatic joint degradation was monitored in-vivo using MMPSense750 reagent.

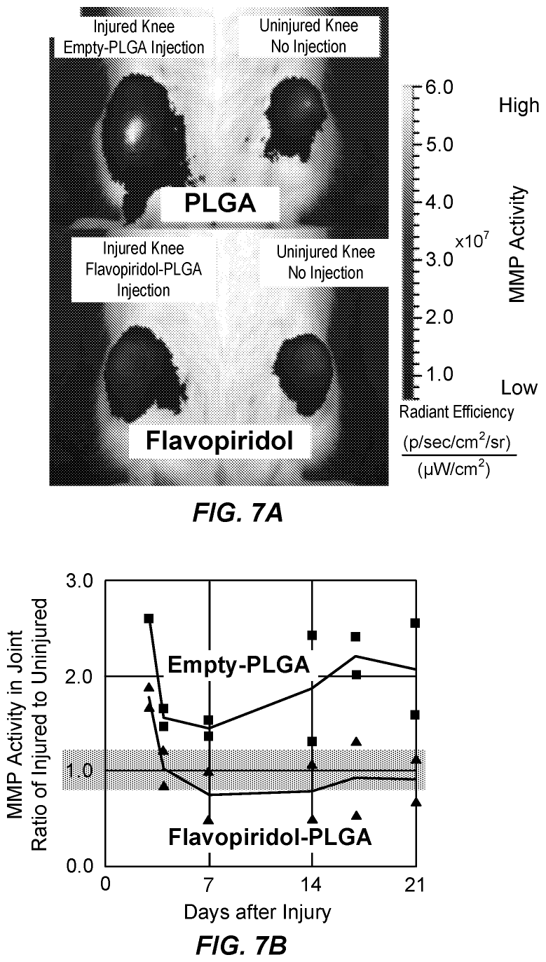

MMPSense750 becomes fluorescent in the presence of local MMP activity, and the ACL-rupture injury causes a robust increase in fluorescence in untreated knees and in knees with empty PLGA microparticles. This injury-induced MMP activity becomes detectable within days of injury, and remains elevated in the injured joints for at least 3 weeks. However, in knees with PLGA-encapsulated flavopiridol, the MMPSense750 signal did not increase after injury. This indicates that the single intra-articular injection of flavopiridol-releasing PLGA microparticles effectively prevented MMP activity in the injured joints, and that the benefits of the single injection lasted for at least 3 weeks (see FIG. 7A). This is consistent with sustained inhibition of CDK9 activity, as we have previously shown that in this model, in-vivo MMP activity is dependent on the transcriptional activation of primary response genes and can be inhibited with repeated systemic administrations of flavopiridol.

### Example 4. Comparative Synthesis of PLGA-Flavopiridol Particles with Altered Release Profiles

(A) Two lots of PLGA-flavopiridol microparticles were made from a high-viscosity ester-terminated polymer (Purasorb® 5010, LG ratio 50:50, IV=1.0). As compared to the acid-terminated PLGA microparticles (see e.g., Table 1), the ester-terminated form incorporates less CDK9 inhibitor into the microparticles. As shown in Table 2, the loading percentage was unacceptably low (0.14% to 0.17%) and thus not viable for commercialization.

| Lot number | 41227 | 53002 |
|---|---|---|
| Ave. particle size | 15.24 ± 6.584 μm | 12.47 ± 6.584 μm |
| D10 | 5.884 μm | 5.374 μm |
| D50 | 15.63 μm | 11.90 μm |
| D90 | 23.94 μm | 20.24 μm |
| Loading | 0.14% | 0.17% |

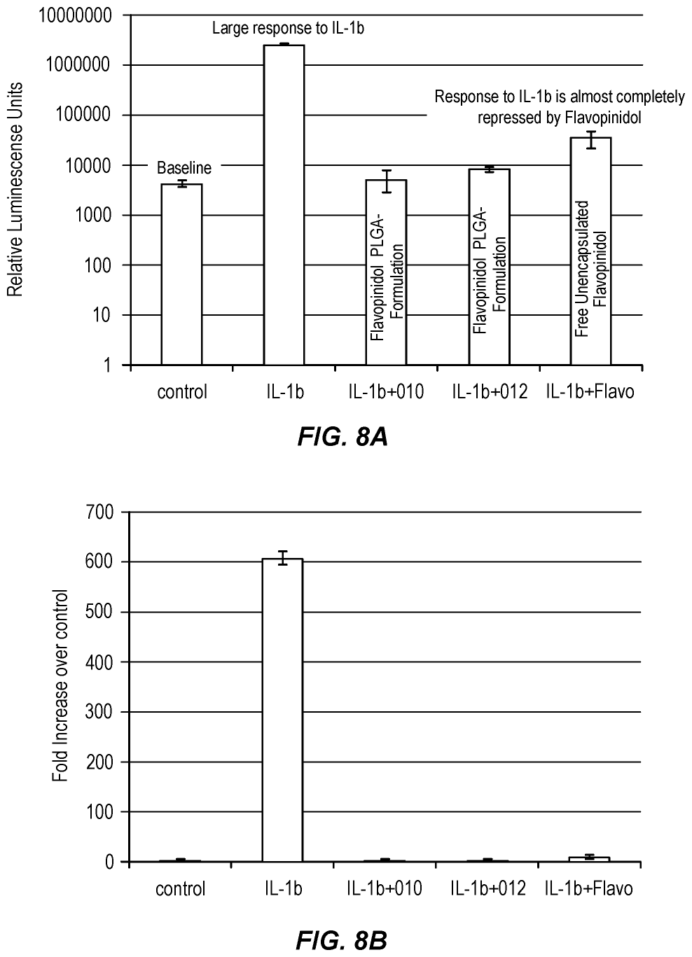

FIGS. 8-10 show comparative particles of PLGA/flavopiridol with altered loading and/or release profiles. FIG. 8 shows a population of microparticles having lower levels of loading and release of flavopiridol. These microparticles were made with ester-terminated PLGA and had a higher inherent viscosity (1.0 dL/g) as compared to microparticles formulated with acid terminated PLGA having an inherent viscosity generally equal to or less than about 0.75 dL/g.

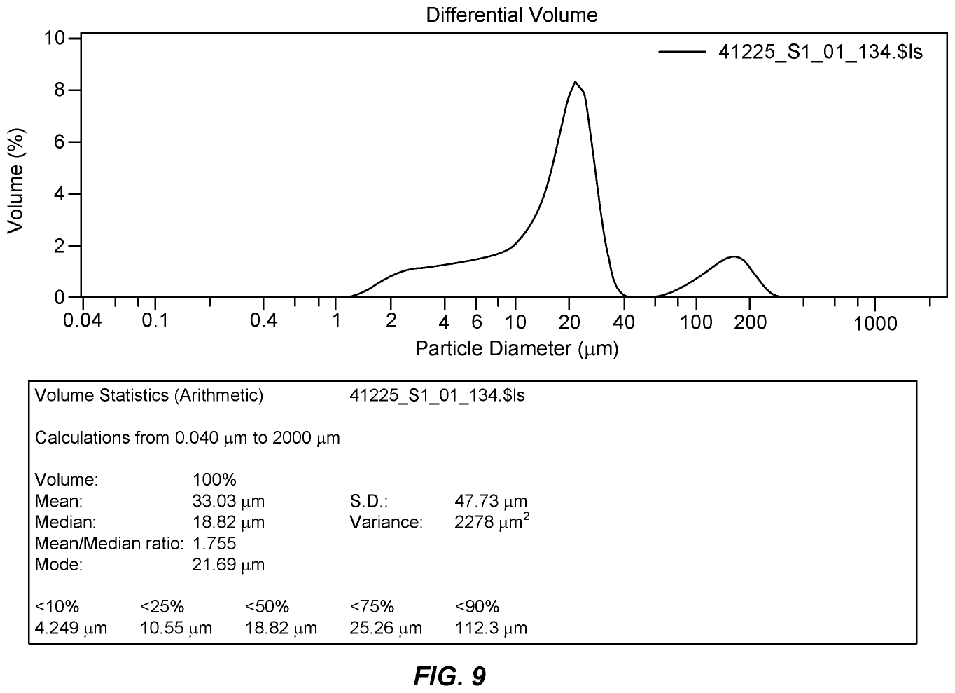

FIG. 9 shows a microparticle formulations where the release of the CDK9 inhibitor was limited to about 60-75% of the CDK9 inhibitor, where the microparticles do not reach an 80% release. These microparticles were formulated with an ester-terminated PLGA, an inherent viscosity of 1.0 dL/g and further exhibited clumping.

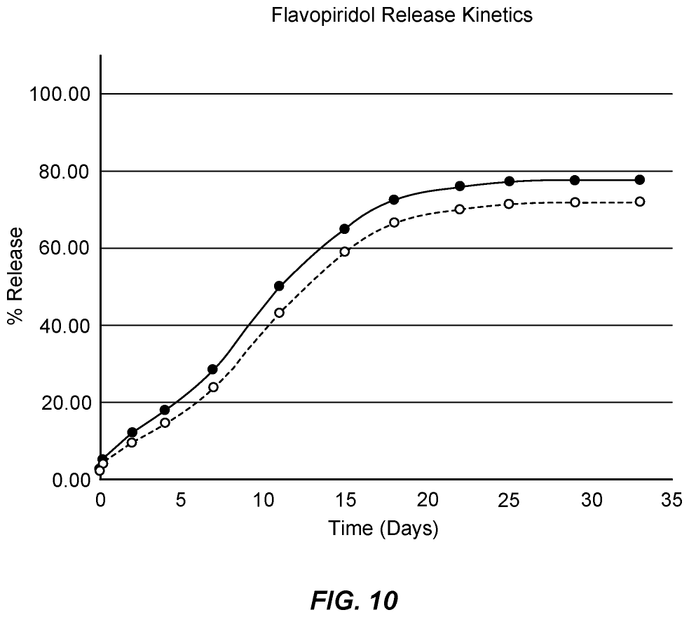

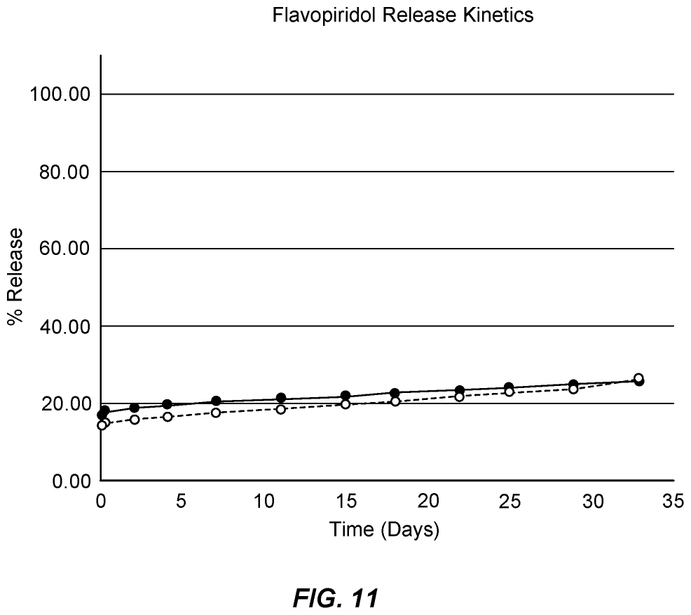

FIG. 10 shows two microparticle formulations of acid terminated PLGA with an inherent viscosity between 0.7-0.9 dL/g. These microparticles exhibited loading of the flavopiridol, but released an initial burst of CDK9 inhibitor, about 20% of the inhibitor, with no further release of inhibitor over the time period shown. A summary comparing loading and release efficiencies of flavopiridol in various PLGA formulations is shown in Table 1 below.

TABLE 1
Formulation Characteristics

| | | | LG | IV | Loading | Loading | Size | Suitable or |
|---|---|---|---|---|---|---|---|---|
| | Polymer | Termination | Ratio | (dL/g) | Efficiency | (wt/wt) | (μm) | unfavorable |
| A | Lactel B6013-2 | Acid | 50/50 | 0.55-0.75 | 65.5% | 1.31% | 15.87 | Suitable |
| B | Lactel B6013-2 | Acid | 50/50 | 0.55-0.75 | 58.0% | 1.16% | 14.14 | Suitable |
| C | Purasorb 5004A | Acid | 50/50 | 0.4 | 65.5% | 1.31% | 14.05 | Suitable |
| D | Purasorb 5004A | Acid | 50/50 | 0.4 | 74.5% | 1.49% | 14.68 | Suitable |
| E | Purasorb 5010 | Ester | 50/50 | 1.0 | 7.0% | 0.14% | 15.24 | (1) |
| F | Lactel B6013-2 | Acid | 50/50 | 0.55-0.75 | 19.0% | 0.38% | 11.11 | (2)(3) |
| G | Lactel B6013-2 | Acid | 50/50 | 0.55-0.75 | 21.0% | 0.42% | 13.59 | Suitable |
| H | Purasorb 5010 | Ester | 50/50 | 1.0 | 8.5% | 0.17% | 12.47 | (1) |
| I | Lactel B6012-4 | Acid | 75/25 | 0.7-0.9 | 34.0% | 0.68% | 9.0 | (2) |
| J | Lactel B6013-2 | Acid | 50/50 | 0.55-0.75 | 79.0% | 1.58% | 16.21 | Suitable |
| K | Lactel B6012-4 | Acid | 75/25 | 0.7-0.9 | 21.0% | 0.41% | 12.68 | Suitable |
| L | Purasorb 5004A | Acid | 50/50 | 0.4 | 56.5% | 1.13% | 16.72 | Suitable |
| M | Lactel B6012-4 | Acid | 75/25 | 0.7-0.9 | 25.5% | 0.51% | 14.84 | (4) |
| N | Lactel B6012-4 | Acid | 75/25 | 0.7-0.9 | 19.8% | 0.79% | 16.33 | (4) |
| O | Purasorb 5010 | Ester | 50/50 | 1.0 | | | | (3) |
| P | Lactel B6013-2 | Acid | 50/50 | 0.55-0.75 | 67.0% | 1.34% | 15.1 | Suitable |
| Q | Lactel B6013-2 | Acid | 50/50 | 0.55-0.75 | 57.5% | 1.15% | 18.95 | Suitable |
| R | Lactel B6013-2 | Acid | 50/50 | 0.55-0.75 | 51.5% | 1.03% | 17.15 | Suitable |
| S | Lactel B6013-2 | Acid | 50/50 | 0.55-0.75 | 59.5% | 1.19% | 14.86 | Suitable |

(1) = inadequate loading;
(2) = unsuitable particle size or size distribution;
(3) = aggregation;
(4) = unsuitable release characteristics

### Example 5. Administration of Formulated Flavopiridol to Equine Subjects

Drug preparation: Flavopiridol doses (0.122 mg) were embedded in 10.26 mg of PLGA microparticles. The microparticles were resuspended in 2 mL of sterile saline for injection.

(A) Surgical cases: A total of 60 horses were studied, fifty-two with condylar fractures, and 8 with first phalangeal (P1) fractures. The horses were divided into 2 groups, with 36 treated and 24 controls (saline alone). Surgical cases were randomized during surgery into treated and control groups. The surgeon was provided with a prepared syringe, and patient horses were treated immediately after lag screw compression of intraarticular fractures. All horses were treated postoperatively with identical antibiotics and NSAIDs, and were hand-walked daily to assess comfort level. At bandage change, the limbs were assessed for intraarticular effusion, edema, incision discharge, and pain on flexion. The surgical site was radiographed monthly to assess fracture healing.

Comfort scores were similar between the treated and control groups in the condylar fractures, but were significantly improved in the fractures of P1. Across both groups, effusion scores were markedly improved in the treated group beginning at 24 hours postoperatively. No significant difference was noted in the edema scores, although within this group of horses the edema was primarily centered around the stab incisions for lag screw insertion.

The treated horses at time points greater than 90 days postoperative demonstrated marked improvement in range of motion, effusion scores, and comfort. Radiographically, no significant difference was noted in the rate of healing between the treated and untreated controls, indicating that the inhibition of the inflammatory response does not negatively impact healing.

(B) Training cases (athletic horses in competition): Horses with performance-limiting lameness issues referable to the metacarpophalangeal and metatarsophalangeal joints and the carpal joints were treated with the microparticles described above.

Cases were assessed pre-injection with radiography, computerized tomography, and ultrasonography to determine the extent of preexisting disease. Cases were classified as having preexisting osteochondral fragments (n=18), osteophytosis (n=65), partial collapse of the joint (n=3), extensive subchondral cystic lesions (n=1), and moderate to severe subchondral remodeling (n=15). The horses were examined daily for lameness grade, effusion, and response to flexion. A total of 206 injections were administered. No adverse reactions were experienced.

In cases with only synovitis, synovial effusion reduced on average 25% within the first 24 hours, decreased by 75% at 48 hours and were normal by 60-72 hours. Comfort scores improved within 36 hours of injection. On average, horses with preexisting arthritic signs demonstrated improvement in the clinical scores for 3-4 weeks from injection.

Several horses were injected repeatedly every 30 days from inception of the trial. Clinical examination of the synovial fluid revealed improved viscosity and reduction in total protein scores in all cases. No increase in the severity of the radiographic or tomographic abnormalities were noted in these cases with repeated treatments.

### Example 6. Formulation of SNS-32, Voruciclib, and Dinaciclib

(A) Preparation: Encapsulation of CDK-9 inhibitors SNS-032 (a non-flavonoid), voruciclib (a flavonoid), and dinaciclib (a non-flavonoid) in PLGA or polycaprolactone (PCL) was performed as follows. Preparation of poly(lactic-co-glycolic) acid (PLGA) or polycaprolactone (PCL) particles with CDK9 inhibitors was performed using a single emulsion-solvent evaporation technique. Briefly, 500 mg of PLGA or PCL was dissolved in 2.5 mL of 2% v/v dimethylsulfoxide in methylene chloride (for PLGA) or chloroform (for PCL). CDK9 inhibitors (2% g/g polymer, dinaciclib, SNS-032, and voruciclib) were added to the polymer solutions, and then added to 5 mL of 10% aqueous poly(vinyl alcohol). The solutions were vortexed at full speed (30 seconds for PLGA or 45 seconds for PCL) to form microparticles. The microparticle suspensions were transferred to 150 mL of 1% poly(vinyl alcohol), and stirred for 24 hours. The particles were pelleted, washed with water, lyophilized and stored at −20° C. for further use. Size distribution was measured using an AccuSizer model 770 optical particle sizer (Particle Sizing Systems). Results are shown in Table 4 below.

TABLE 2
Microparticle loading efficiency

| | % loading efficiency | |
|---|---|---|
| CDK9 inhibitor: | PLGA | PCL |
| Dinaciclib | 3.6 | 0.6 |
| SNS-032 | 30.7 | 0.3 |
| Voruciclib | 59.9 | 1.1 |

Table 4 shows the percentage of the CDK9 inhibitor (i.e., the percentage of the 2 g) that is loaded into microparticles using each of the CDK9 inhibitors with either PLGA or PCL. For example, SNS-032 has a 30.7% loading efficiency, such that of the 2 g of starting SNS-032, about 0.61 g was encapsulated in the PLGA microparticles. Lower amounts of drug encapsulation result in less inhibitor per microparticle. If the loading efficiency decreases below a certain threshold, the amount of microparticles required to deliver a therapeutic dose of the CDK9 inhibitor can become prohibitive (for cost, efficiency, injection volume, and potentially, an adverse reaction of the treated subject to the administered microparticles). The results show that PCL failed to incorporate an adequate amount of CDK9 inhibitor, and that PLGA failed to incorporate an adequate amount of the non-flavonoid inhibitor dinaciclib.

(B) Release: The release characteristics of the microparticles prepared above were determined using the procedure set forth in Example 2. The results are shown in Table 3 below.

TABLE 3
Release of CDK9 inhibitors from different microparticle formulations.

| Day | Dinaciclib | SNS-032 | Voruciclib |
|---|---|---|---|
| | PLGA (% release) | | |
| 1 | 0.6 | 4.0 | 3.4 |
| 3 | 1.5 | 5.4 | 4.8 |
| 5 | 2.2 | 7.0 | 6.5 |
| | PCL (% release) | | |
| 3 | 0 | 0 | 0.36 |
| 7 | 0 | 0 | 0.43 |

The results demonstrate that microparticles containing dinaciclib fail to release the compound at an adequate rate for treatment, and that microparticles using PCL fail to release any CDK9 inhibitor tested at an adequate rate for treatment.

Table 4 below compares the loading and release of CDK9 inhibitors and formulation agents in PLGA and PCL

TABLE 4
Loading and Release Efficiency

| CDK9 inhibitor | PLGA loading | PLGA release | PLC loading | PLC release |
|---|---|---|---|---|
| Flavopiridol | +++ | +++ | | |
| Voruciclib | +++ | +++ | − | +/− |
| Dinaciclib | − | + | − | − |
| SNS-032 | ++ | +++ | − | − |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A microparticle comprising a cyclin-dependent kinase 9 (CDK9) inhibitor and poly(lactic-co-glycolic) acid (PLGA), wherein the CDK9 inhibitor is encapsulated by the PLGA, and wherein the microparticle has a diameter of from about 3 to about 100 microns and provides a sustained release of the CDK9 inhibitor.

2. The microparticle of claim 1, wherein the CDK9 inhibitor is selected from the group consisting of flavopiridol, SNS-032, voruciclib and a derivative thereof, or pharmaceutically acceptable salt thereof.

3. (canceled)

4. The microparticle of claim 1, wherein the CDK9 inhibitor is flavopiridol.

5. The microparticle of claim 4, wherein the PLGA has a lactic acid to glycolic acid (L:G) ratio of about 50:50 to about 75:25.

6. (canceled)

7. (canceled)

8. (canceled)

9. (canceled)

10. The microparticle of claim 1, wherein the microparticle releases the CDK9 inhibitor over a duration of about 60 days following administration.

11. The microparticle of claim 5, wherein the microparticle releases from about 3% to about 30%, about 3% to about 20%, over 24 hours following administration.

12. (canceled)

13. (canceled)

14. (canceled)

15. (canceled)

16. (canceled)

17. (canceled)

18. (canceled)

19. (canceled)

20. The microparticle of claim 11, wherein the microparticle releases from about 80% to about 95% of the CDK9 inhibitor over 30 days following administration.

21. A pharmaceutical composition comprising a plurality of microparticles of claim 1 and a pharmaceutically acceptable carrier.

22. The pharmaceutical composition of claim 21, wherein the plurality of microparticles has a mean diameter of from about 10 to about 20 microns.

23. (canceled)

24. (canceled)

25. The pharmaceutical composition of claim 21, wherein 90% of the mass of the plurality of microparticles (D90) has a diameter of less than about 30 microns.

26. The pharmaceutical composition of claim 21, wherein the plurality of microparticles has from about 0.5% to about 5% by weight of the CDK9 inhibitor.

27. A method of treating a subject in need thereof, comprising administering a therapeutically effective amount of a plurality of microparticles, the microparticles comprising a CDK9 inhibitor and a poly(lactic-co-glycolic) acid (PLGA), wherein the CDK9 inhibitor is encapsulated by the PLGA, and wherein the microparticles provide a sustained release of the CDK9 inhibitor.

28. (canceled)

29. (canceled)

30. (canceled)

31. The method of claim 27, wherein the CDK9 inhibitor is flavopiridol.

32. (canceled)

33. (canceled)

34. (canceled)

35. The method of claim 27, comprising administering a pharmaceutical composition of claim 21, at a site of an inflammation in the subject.

36. The method of claim 35, wherein the subject has a disease or condition selected from arthritis, osteoarthritis, post-traumatic osteoarthritis, and a traumatic injury.

37. The method of claim 36, wherein the site of inflammation is a joint, cartilage, or a site of traumatic injury.

38. (canceled)

39. The method of claim 36, wherein the pharmaceutical composition is administered by injection.

40. A method of treating a site of inflammation comprising, administering to the site a composition comprising a CDK9 inhibitor formulated into a plurality of microparticles, wherein the microparticles provide a sustained release of the CDK9 inhibitor at the site for at least 24 hours, and whereby inflammation at the site is thereby reduced or ameliorated.

41. (canceled)

42. The method of claim 40, wherein the CDK9 inhibitor is flavopiridol.

43. (canceled)

44. The microparticle of claim 11, wherein the microparticle releases the CDK9 inhibitor over a duration of about 60 days following administration.

45. The pharmaceutical composition of claim 21, wherein the CDK9 inhibitor is flavopiridol.

46. The pharmaceutical composition of claim 21, formulated for delivery by injection.
